(19) United States
(12) Patent Application Publication
(10) Pub. No.: US 2026/0355197 A1
(43) Pub. Date: Nov. 12, 2026

(54) METHOD FOR TREATING ATOPIC DERMATITIS

(71) Applicant: BEIJING INNOCARE PHARMA TECH CO., LTD., Beijing (CN)

(72) Inventors: Jisong CUI, Beijing (CN); Wei ZHOU, Shanghai (CN); Bin ZHANG, Beijing (CN); Renbin ZHAO, Beijing (CN)

(21) Appl. No.: 19/703,646

(22) Filed: Jun. 10, 2026

Related U.S. Application Data
(63) Continuation of application No. PCT/CN2024/138717, filed on Dec. 12, 2024.
(60) Provisional application No. 63/617,161, filed on Jan. 3, 2024.

(30) Foreign Application Priority Data
Dec. 15, 2023 (CN) 202311735481.6

(51) Int. Cl. A61K 31/506; A61P 17/00

(52) U.S. Cl. CPC A61K 31/506; A61P 17/00

(57) ABSTRACT

The present invention is directed to a method for treating atopic dermatitis by administering to a subject in need thereof Compound A, B, or C. Oral and topical are preferred routes of administration. In one embodiment, the subject is treated with about 50-150 mg/day of Compound A, B, or C by oral administration.

### CROSS-REFERENCE TO RELATED APPLICATION(S)

This application is a continuation of PCT/CN2024/138717, filed Dec. 12, 2024; which claims the benefit of U.S. Provisional Application No. 63/617,161, filed Jan. 3, 2024, and Chinese Patent Application CN202311735481.6, filed Dec. 15, 2023. The contents of the above-identified applications are incorporated herein by reference in their entirety.

### TECHNICAL FIELD

The present invention relates to a method for treating atopic dermatitis by administering to a subject in need thereof Compound A, B, or C. One preferred route of administration is oral administration.

### BACKGROUND

Atopic dermatitis, often referred to as eczema, is a chronic (long-lasting) disease that causes inflammation, redness, and irritation of the skin. It is a common condition that usually begins in childhood; however, anyone can get the disease at any age. Atopic dermatitis causes the skin to become extremely itchy. Scratching leads to further redness, swelling, cracking, “weeping” clear fluid, crusting, and scaling. In most cases, there are periods of time (flares) when the disease is worse, followed by remissions when the skin improves or clears up entirely.

The most common symptom of atopic dermatitis is itching, which can be severe. Other common symptoms include red, dry patches of skin; rashes that may ooze or weep clear fluid; bleeding when scratched; and thickening and hardening of the skin. The symptoms can flare in multiple areas of the body at the same time and can appear in the same locations and in new locations. The appearance and location of the rash vary depending on age; however, the rash can appear anywhere on the body.

### BRIEF DESCRIPTION OF THE DRAWINGS

FIG. 1 shows the clinical trial design of Example 1.

FIG. 2 shows Eczema Area and Severity Index (EASI) Total Scores. The left panel shows percent change from baseline in EASI total score at Week 4. The right panel shows % change from baseline in EASI total score at Week 1, 2, and 4.

FIG. 3 shows EASI 50, EASI 75, EASI 90, IGA 0/1 of placebo, ICP-332 80 mg, and ICP-332 120 mg results at Week 4.

FIG. 4 shows EASI-75 (difference from placebo) of ICP-332 versus those of other drugs for treating AD as monotherapy. The treatment period for ICP-332 was 4 weeks, while it was 12 or 16 weeks for other drugs with their approved recommended doses from phase III studies. The data of other drugs are based on available published literature.

### DETAILED DESCRIPTION OF THE INVENTION

The inventors have discovered that Compound A, B, or C at a dosage of 50-150 mg/day, for example, 80 mg/day and 120 mg/day, is effective in treating atopic dermatitis by oral administration.

The chemical structures of compound A, B, and C are shown below. Compound A is referred to as ICP-332 in this application.

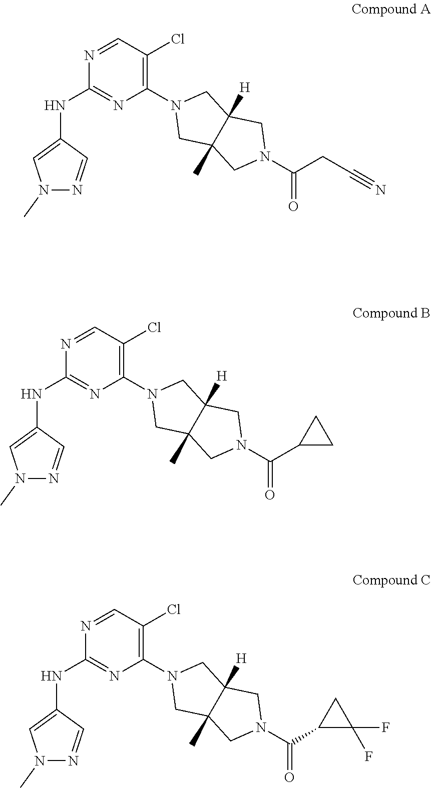 [SMILES: Cn1cc(Nc2ncc(Cl)c(N3C[C@@H]4CN(C(=O)CC#N)C[C@]4(C)C3)n2)cn1 ; Cn1cc(Nc2ncc(Cl)c(N3C[C@@H]4CN(C(=O)C5CC5)C[C@]4(C)C3)n2)cn1 ; Cn1cc(Nc2ncc(Cl)c(N3C[C@@H]4CN(C(=O)[C@@H]5CC5(F)F)C[C@]4(C)C3)n2)cn1]

### Pharmaceutical Compositions

The present disclosure provides pharmaceutical compositions comprising one or more pharmaceutically acceptable carriers and an active compound of Compound A, B, or C. The active compound in the pharmaceutical compositions in general is in an amount of about 0.1-5% for an injectable formulation, about 1-90% for a tablet formulation, and 1-100% for a capsule formulation, about 0.01-20%, 0.05-20%, 0.1-20%, 0.2-15%, 0.5-10%, or 1-5% (w/w) for a topical formulation, and about 0.1-5% for a patch formulation.

In one embodiment, the pharmaceutical composition can be in a dosage form such as tablets, capsules, granules, fine granules, powders, syrups, suppositories, injectable solutions, patches, or the like. For example, the active compound can be prepared in Ora-Plus® (an aqueous-based oral suspending vehicle including water, microcrystalline cellulose, carboxymethylcellulose, xanthan gum, carrageenan, calcium sulfate, and trisodium phosphate). The above pharmaceutical composition can be prepared by conventional methods.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, and gels. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositol; poloxamers and poloxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as microcrystalline cellulose, methylcellulose, hydroxypropyl cellulose, hydroxyethyl cellulose, carboxymethyl cellulose, and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate; xanthan gum, and carrageenan. Such pharmaceutically acceptable carriers may be preserved against bacterial contamination using well-known preservatives, these include, but are not limited to, benzalkonium chloride, ethylenediaminetetraacetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of the active compound may contain other excipients that have no bioactivity and no reaction with the active compound. Excipients of a tablet or a capsule may include fillers, binders, lubricants and glidants, disintegrators, wetting agents, and release rate modifiers. Binders promote the adhesion of particles of the formulation and are important for a tablet formulation. Examples of excipients of a tablet or a capsule include, but not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone. For example, a tablet formulation may contain inactive ingredients such as colloidal silicon dioxide, crospovidone, hypromellose, magnesium stearate, microcrystalline cellulose, polyethylene glycol, sodium starch glycolate, and/or titanium dioxide. A capsule formulation may contain inactive ingredients such as gelatin, magnesium stearate, and/or titanium dioxide.

Topical formulations including the active compound can be in the form of gel, cream, lotion, liquid, emulsion, ointment, spray, solution, and suspension. The inactive ingredients in the topical formulations may include emollient, permeation enhancer, solubility enhancer, and rheology/texture modifier. The inactive ingredients for example include, but not limited to, lauryl lactate, diethylene glycol monoethylether, DMSO, silicone elastomer, caprylic/capric triglyceride, octisalate, silicone fluid, squalene, sunflower oil, and silicone dioxide.

For example, a patch formulation may comprise the active compound and some inactive ingredients such as 1,3-butylene glycol, dihydroxyaluminum aminoacetate, disodium edetate, D-sorbitol, gelatin, kaolin, methylparaben, polysorbate 80, povidone, propylene glycol, propylparaben, sodium carboxymethylcellulose, sodium polyacrylate, tartaric acid, titanium dioxide, and purified water. A patch formulation may also contain skin permeability enhancer such as lactate esters (e.g., lauryl lactate) or diethylene glycol monoethylether.

### Method of Treating Atopic Dermatitis

The present invention is directed to a method of treating atopic dermatitis. Compounds A, B, or C can be administered in the form of a pharmaceutical composition that additionally contains a pharmaceutically acceptable carrier. The method comprises the steps of identifying a subject suffering from atopic dermatitis and administering to the subject an effective amount of Compound A, B, or C to treat atopic dermatitis. “An effective amount” as used herein, is the amount effective to treat atopic dermatitis by ameliorating the pathological condition or reducing the symptoms of the disease.

A pharmaceutical composition comprising Compound A, B, or C can be applied by local administration and systemic administration. Local administration includes topical administration. Systemic administration includes oral, parenteral (such as oral, intravenous, intramuscular, subcutaneous or rectal), and other systemic routes of administration. In systemic administration, the active compound first reaches plasma and then distributes into target tissues. Oral administration is a preferred route of administration for the present invention. Those of skill in the art will recognize that a wide variety of delivery mechanisms may be suitable for the present invention.

Dosing of the composition can vary based on the extent of the injury and each patient's individual response, and the possibility of co-usage with other therapeutic treatments including use of other therapeutic agents.

In general, an effective dose of A, B, or C to be orally administered to a human subject is about 50-150 mg per day. The daily dosage may be administered in one administration or in separate administrations of 2, 3, 4, or 6 equal doses. For example, the dosage is about 50 mg once a day, 80 mg once a day, 100 mg once a day, or 120 mg once a day. The dosage may also be about 50 mg twice a day, 60 mg twice a day, or 70 mg twice a day.

In one embodiment, the pharmaceutical composition is administrated subcutaneously to the subject.

The time period that the subject is dosed with Compound A, B, or C in any of the methods described above can range, for example, from about at least 1 day, at least 3 to 5 days, at least 1 week, or at least one month. In one embodiment, the treatment for moderate atopic dermatitis is 3-6 months. In one embodiment, the treatment for moderate atopic dermatitis is up to one year.

In one embodiment, the subject is orally treated with about 50-150 mg/day of Compounds A, B, or C.

In one embodiment, the subject is orally treated with about 50-150 mg of Compounds A, B, or C once daily.

In one embodiment, the subject is orally treated with about 80 mg or about 120 mg of Compounds A, B, or C once a day (qd).

“About” as used herewith, refers to ±10%, preferably ±5%, of the recited value.

In one embodiment, the efficacy of Compound A, B, or C in treating atopic dermatitis in patients is evaluated by whether the treatment reduces the symptoms of the disease such as red, dry patches of skin; rashes that ooze or weep clear fluid; bleeding when scratched; and thickening and hardening of the skin.

In one embodiment, the efficacy of Compound A, B, or C in treating atopic dermatitis in patients is evaluated by the Eczema Area and Severity Index (EASI) total score. EASI is a validated scale and can be used reliably in the assessment of severity and extent of atopic dermatitis. The total EASI score ranges from 0 to 72 points, with the highest score indicating worse severity of atopic dermatitis.

In one embodiment, the efficacy of Compound A, B, or C in treating atopic dermatitis in patients is evaluated by SCORAD (scoring atopic dermatitis). SCORAD is a clinical tool used to assess the extent and severity of eczema. SCORAD adds up the scores of the affected areas (head and neck, upper limbs, lower limbs, anterior trunk, back and genitals), as a percentage of the whole body. In each area, the intensity of each of the signs (redness, swelling, oozing/crusting, scratch marks, skin thickening (lichenification), and dryness are assessed as none (0), mild (1), moderate (2) or severe (3).

In general, an effective dose of Compound A, B, or C, when orally administered to a human subject, is about 50-150 mg per day. The daily dosage may be administered in one administration or in separate administrations of 2, 3, 4, or 6 equal doses.

The present invention is also directed to Compound A, B, or C, for use in a method of treating atopic dermatitis, wherein it is orally administered to a patient.

The present invention is further directed to use Compound A, B, or C, for preparing a medicament for treating atopic dermatitis.

The present invention is useful in treating a mammal subject, such as humans, horses, and dogs. The present invention is particularly useful in treating humans.

### EXAMPLE

### Example 1. Clinical Study Protocol of Orally Administered Compound A in Atopic Dermatitis Patients

### Trial Design

This is a randomized, double-blind, placebo-controlled, phase 2 study to evaluate the safety, efficacy, pharmacokinetics, and pharmacodynamics of ICP-332 (Compound A) in patients with moderate to severe atopic dermatitis.

The study consists of a 35-day screening period, a 4-week double-blind treatment period, and a 28-day safety follow-up period. Investigators, subjects, and the sponsor study team will be blinded throughout the study.

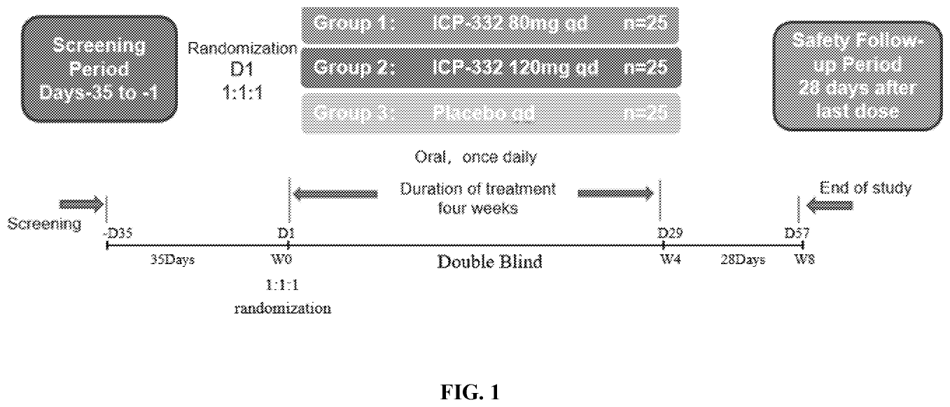

FIG. 1 shows the clinical trial design. Subjects are randomized in the ratio of 1:1:1 to one of two treatment groups or placebo. All subjects receive the study drug once daily for 4 weeks. Randomization is stratified by intensive PK blood sampling (yes vs. no).

A total of 75 subjects with 25 subjects in each group are enrolled.
(1) ICP-332 80 mg, QD for 28 days (40 mg*2 tablets+Placebo*1 tablet)
(2) ICP-332 120 mg. QD for 28 days (40 mg*3 tablets)
(3) Placebo. QD for 28 days (Placebo*3 tablets)

### Criteria for Inclusion

Subjects must meet all of the following criteria to be enrolled:
1. Male or female subjects aged≥18 years and ≤75 years.
2. Subject has a clinical diagnosis of atopic dermatitis or eczema for at least 1 year prior to Day 1 and has confirmed atopic dermatitis (as per the Williams criteria) at the screening visit.
3. Subject meets the criteria for moderate to severe AD, defined as EASI score≥16, affected body surface area (BSA)≥10%, and IGA score≥3, as assessed by the investigator, at screening and baseline.
4. Documented history of inadequate response to topical corticosteroids (TCSs) or topical calcineurin inhibitors (TCIs) (treatment for at least 4 weeks within 1 year prior to the screening visit), or for whom other topical treatments are otherwise medically inadvisable (e.g., due to important side effects or safety risks).
5. Able and willing to use bland emollients without additives twice daily from at least 7 days prior to baseline and throughout the study.
6. Females must have a negative serum pregnancy test at the screening visit, and females of childbearing potential must have a negative urine pregnancy test at the baseline visit prior to the first study drug dosing.
7. Having voluntarily signed and dated the Informed Consent Form (ICF) prior to the initiation of any screening or study-specific procedures.
8. Willing and able to comply with scheduled visits, treatment plan, laboratory tests, and other study procedures.
9. Women of childbearing potential (WOCBP) must have had menstruation during the screening period and must agree to use a supplemental barrier method combined with a highly effective method of contraception (according to ICH guideline M3 [R2]) during this study and for 90 days after the last dose of study drug.

### Main Criteria for Exclusion

Participants are excluded from the study if any of the following criteria apply:
1. Pregnant or breastfeeding females.
2. Have a history of active skin diseases or skin infections (bacterial, fungal, or viral) requiring systemic treatment or would interfere with appropriate assessment of AD lesions within 4 weeks prior to the baseline visit.
3. Current evidence or history of infection, including:
   History (single episode) of disseminated herpes zoster or disseminated herpes simplex, or a recurrent (more than one episode of) localized, dermatomal herpes zoster.
   History of known invasive infection (e.g., listeriosis and histoplasmosis).
   Chronic recurring infection and/or active invasive infection (e.g., listeriosis and histoplasmosis) and viral infection make the subject unsuitable for the study based on clinical assessment by the investigator.
   Have a known immunodeficiency syndrome.
   Presence of active tuberculosis or a positive test for tuberculosis.
   Non-skin related active infection(s) requiring treatment with parenteral anti-infectives within 30 days or with oral anti-infectives within 14 days prior to the baseline visit.
4. Active HBV, HCV or HIV infection, and syphilis infection, defined as:
   HBV: hepatitis B surface antigen (HBsAg) positive (+) or detected sensitivity on the HBV DNA polymerase chain reaction (PCR) qualitative test for hepatitis B core antibody (HBc Ab) positive (+) subjects.
   HCV: history of or positive for hepatitis C virus (HCV) antibody at screening. However, if a participant with a history of HCV infection has completed standard treatment at least 1 year prior to screening and is HCV RNA-negative by PCR at screening, this participant will not be excluded from the study.
   HIV: confirmed positive anti-HIV antibody (HIVAb) test.
   Anti-Treponema pallidum antibody test results are abnormal and clinically significant as judged by the investigator.
5. Presence of underlying medical conditions or problems, including but not limited to the following:
   Clinically relevant or significant ECG abnormalities including QT interval corrected for heart rate (QTc) using Fridericia's formula (QTcF)>500 msec.
   A history of moderate to severe congestive heart failure (New York Heart Association Class III or IV), recent (within the past 6 months) cerebrovascular accident, myocardial infarction, or coronary artery stenting, or uncontrolled hypertension (a confirmed systolic blood pressure>160 mmHg or diastolic blood pressure>100 mmHg).
   Subject has been a previous recipient of an organ transplant.
   History of gastrointestinal perforation, diverticulitis or significantly increased risk of gastrointestinal perforation as judged by the investigator.
   Conditions that may interfere with study drug absorption, including but not limited to short-bowel syndrome.
   Presence of any malignancy within 5 years prior to screening (note: except for completely respected basal cell or squamous cell carcinoma of the skin, or in situ cervix carcinoma that has been treated with no evidence of recurrence).
6. History of any clinically major diseases or clinically significant circulatory system abnormalities, endocrine system abnormalities, nervous system disorders or hematological system disorders, immune system disorders, mental diseases or unstable metabolic disorders, with the exception of atopic dermatitis.
7. Receipt of any following treatment regimens specified within the timeframe outlined below:
   Within 6 months prior to the first dose of study drug:
   Use of any cell-depleting agents, including but not limited to Rituximab, within 6 months or 5 half-lives prior to the first dose of study drug, whichever is longer.
   Within 12 weeks prior to the first dose of study drug:
   Use of any JAK inhibitors.
   Use of other biologic agents within 12 weeks or 5 half-lives prior to the first dose of study drug, whichever is longer.
   Within 8 weeks prior to the first dose of study drug:
   Participation in other studies of investigational drugs within 8 weeks or 5 half-lives prior to the first dose of study drug, whichever is longer.
   Note: any investigational or experimental treatments taken or procedure for AD, psoriasis, psoriatic arthritis, or rheumatoid arthritis in the previous 1 year should be discussed with the Medical Monitor; subjects cannot participate in studies of other investigational or experimental treatments during their participation in the study.
   Within 4 weeks prior to the first dose of study drug:
   Use of oral immunosuppressants (e.g., Cyclosporine A, Azathioprine, Methotrexate, Mycophenolate Mofetil, and Mycophenolate Sodium, systemic Corticosteroids, Interferon-gamma, phosphodiesterase type 4 (PDE4)-inhibitors within 4 weeks or 5 half-lives prior to the first dose of study drug, whichever is longer.
   Phototherapy, and regular use (more than twice a week) of a tanning room/tanning bath, or prolonged sun exposure that may affect disease severity or interfere with disease assessment.
   A dose of any live or live attenuated vaccine within 4 weeks prior to the baseline visit or anticipated requirement for a live or live attenuated vaccine during study participation, including at least 4 weeks after the last dose of study drug. Administration of any inactivated vaccine within 1 week prior to the baseline visit or anticipated need for an inactivated vaccine during study treatment and for at least 1 week following the last dose of study drug.
   Within 1 week prior to the first dose of study drug:
   0 Use of topical treatments that may affect atopic dermatitis (e.g., TCS; TCI; prescription moisturizers or moisturizers containing additives such as ceramides, hyaluronic acid, urea, or filaggrin; antibiotic creams; topical antihistamines).
   Note: patients with stable asthma are allowed to use corticosteroid inhalers and intranasal sprays.
   Traditional Chinese medicines with unknown composition and effects, or known effects on AD
8. An interval of less than 5 half-lives from the last dose of a strong CYP3A inhibitor or inducer (chemical agent, traditional Chinese medicine and dietary supplement) to the first dose of study drug, or a plan to use concurrently medications, dietary supplements or food (e.g., grapefruit or grapefruit juice) with strong CYP3A inhibitory or inductive effect during study participation (see Appendix 5).
9. History of drug or alcohol abuse, as judged by the investigator, within 6 months prior to the baseline visit.
10. Laboratory values meeting the following criteria with the screening period prior to the first dose of study drug (baseline visit):
   Serum aspartate aminotransferase (AST)>2×ULN
   Serum alanine aminotransferase (ALT)>2×ULN
   Total bilirubin≥1.2×ULN
   Estimated glomerular filtration rate (GFR) by the Modification of Diet in Renal Disease (MDRD) study equation<40 mL/min/1.73 m²
   Total white blood cell count (WBC)<2.5×10⁹/L
   Absolute neutrophil count (ANC)<1.5×10⁹/L
   Platelet count<100×10⁹/L
   Absolute lymphocyte count<0.8×10⁹/L
   Hemoglobin (Hb)<10 g/dL
11. Consideration by the Investigator, for any reason, that the subject is an unsuitable candidate to receive ICP-332 or participate in this study.

### Safety Evaluation Variables

Safety evaluation variables include adverse events, vital signs, physical examination, laboratory tests, and electrocardiogram (ECG).

### Pharmacokinetic Sample Collection

Pharmacokinetic (PK) investigation is performed in all subjects in this study, and blood samples are analyzed at the designated bioassay laboratory.

### PK Parameters

Single-dose: maximum plasma concentration (Cmax), time of maximum observed plasma concentration (Tmax), (terminal phase elimination) half-life (T1/2), area under the plasma concentration-time curve (AUC), apparent clearance (CL/F), terminal apparent volume of distribution (Vz/F), etc.

Multiple-dose at steady state: Tmax, T½, AUC, apparent clearance (CL/F), terminal apparent volume of distribution (Vz/F), steady-state trough plasma concentration (Cmin,ss), steady-state maximum plasma concentration (Cmax,ss), average steady-state plasma concentration (Cav,ss), degree of fluctuation (DF), accumulation factor (R), etc.

### Pharmacodynamic Investigation

Pharmacodynamic investigation is performed in all subjects to analyze changes from baseline in pharmacodynamic biomarkers during the treatment. Pharmacodynamic biomarkers include IP-10, hs-CRP, TARC and eosinophils. Serum samples are collected for hs-CRP, IP-10, and TARC testing.

There is a total of 4 visit points for sampling, i.e. on D1, D15, D29 and D57, and the blood sampling time point during administration is within 1 hour pre-dose.

### Duration of Treatment

Four weeks (28 days)

### Duration of Subject Participation in the Study

Thirteen weeks (5-week screening, 4-week treatment, 4-week safety follow-up)

### Clinical Trial Duration

The study consists of a 35-day screening period, a 4-week double-blind treatment period, and a 28-day safety follow-up period. Investigators, subjects, and the sponsor study team are blinded throughout the study.

FIG. 1 shows Phase II design.

### Primary Objective

To evaluate the safety and tolerability of ICP-332 in adult subjects with moderate to severe atopic dermatitis (AD).

### Primary Endpoints

Safety will be assessed by adverse events (AEs), physical examination, vital signs, electrocardiograms (ECGs) and clinical laboratory safety tests.

### Secondary Objective

To evaluate the efficacy of ICP-332 in subjects with moderate to severe AD using the Eczema Area and Severity Index (EASI) total score.
To evaluate the effect of ICP-332 on additional efficacy endpoints and patient reported outcomes over time in adult subjects with moderate to severe AD.
To evaluate the pharmacokinetic (PK) profile of ICP-332 in adult subjects with moderate to severe AD.

### Secondary Endpoints

### Key Secondary Endpoint:

Baseline is defined as the last assessment prior to the first dose.
Percent change from baseline in EASI total score at Week 4.

### Other Efficacy Endpoints

Proportion of subjects achieving Investigator's Global Assessment (IGA) for clear (0) or almost clear (1) at all scheduled timepoints.
Proportion of subjects achieving a ≥50%, 75%, and 90% improvement in the EASI total score (EASI 50, EASI 75, EASI 90) at all scheduled time points.
Percent change from baseline in EASI total score at all scheduled time points except Week 4.
Percent change from baseline in pruritus numerical rating scale (NRS) for at all scheduled time points.
Proportion of subjects achieving ≥3 points improvement in the pruritus NRS from baseline at all scheduled time points.
Proportion of subjects achieving ≥2 points improvement in the IGA from baseline at all scheduled time points.
Change from baseline in affected body surface area (BSA) at all scheduled time points.
Percent change from baseline in SCORing atopic dermatitis (SCORAD) at all scheduled time points.
Proportion of subjects achieving a ≥50%, 75%, and 90% improvement in SCORAD (SCORAD 50, SCORAD 75, SCORAD 90) from baseline at all scheduled timepoints.
Proportion of subjects with Dermatology Life Quality Index (DLQI) total score of “0” or “1” at all scheduled time points.
Change from baseline in DLQI total score at all scheduled time points.

### PK Endpoints

Maximum plasma concentration (Cmax), time of maximum plasma observed concentration (Tmax), (Terminal phase elimination) half-life (T½), area under the plasma concentration-time curve (AUC), apparent clearance (CL/F), terminal apparent volume of distribution (Vz/F), accumulation factor (R), and other steady-state PK parameters, etc.

### Example 2. Clinical Study Results

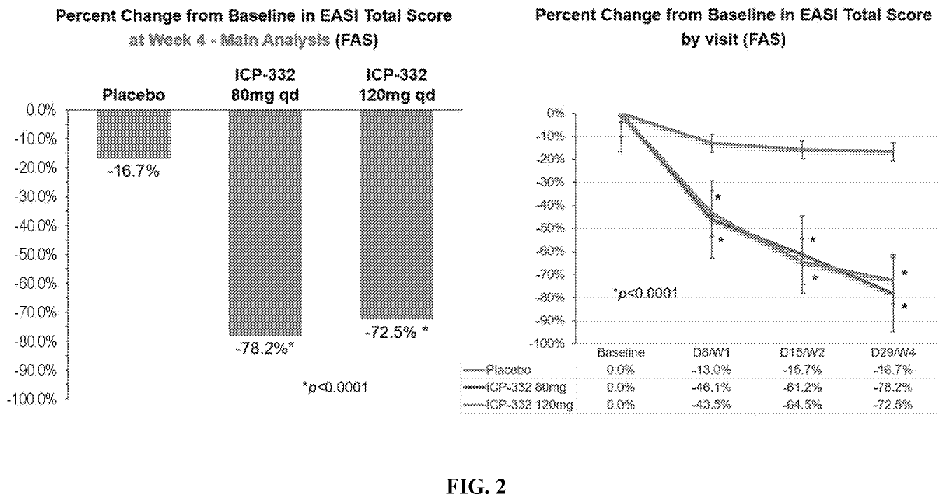

The results of clinical study of Example 1 are summarized in FIG. 2-4.

FIG. 2 shows EASI Total Scores. The left panel shows percent change from baseline in Eczema Area and Severity Index (EASI) total score. EASI is a validated scale and can be used reliably in the assessment of severity and extent of AD. The total EASI score ranges from 0 to 72 points, with the highest score indicating worse severity of AD. The left panel shows that at Week 4, ICP-332 at both 80 mg qd and 120 mg qd significantly reduced the EASI total score (p<0.0001). The right panel shows % change from baseline in EASI total score at Week 1, 2, and 4.

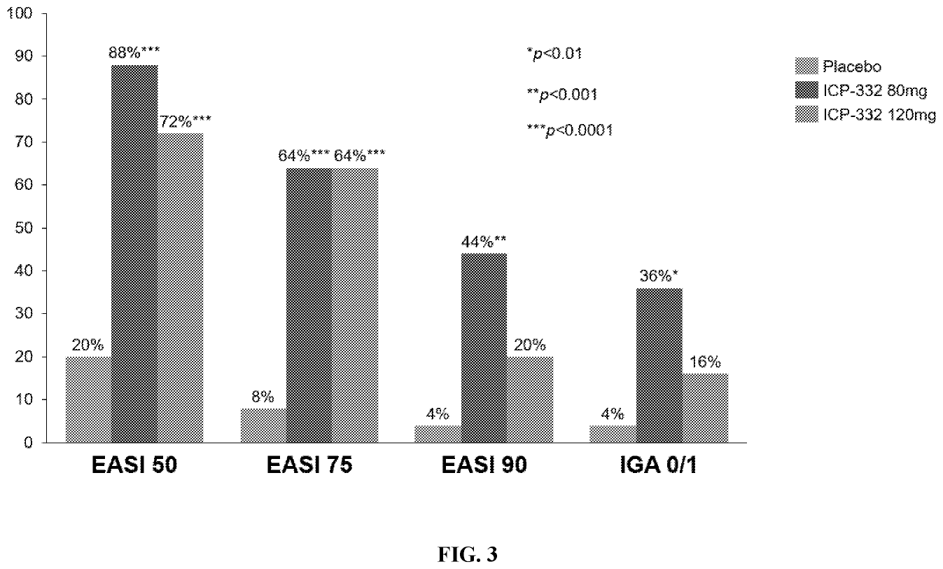

FIG. 3 shows EASI 50, EASI 75, EASI 90, IGA 0/1 of placebo and ICP-332 treated results at Week 4. EASI 50 indicates ≥50% improvement from baseline. EASI 75 indicates ≥75% improvement from baseline. EASI 90 indicates ≥90% improvement from baseline. IGA (Investigator Global Assessment) scale is a five-point scale that provides a global clinical assessment of AD severity by assessing the overall appearance of the lesions at a given time point. IGA scales range from 0 to 4, where 0 indicates clear, 2 is mild, 3 is moderate, and 4 indicates severe AD. A decrease in score relates to an improvement in signs and symptoms. The results show significant improvements at Week 4 by treatment with 80 mg or 120 mg ICP-332.

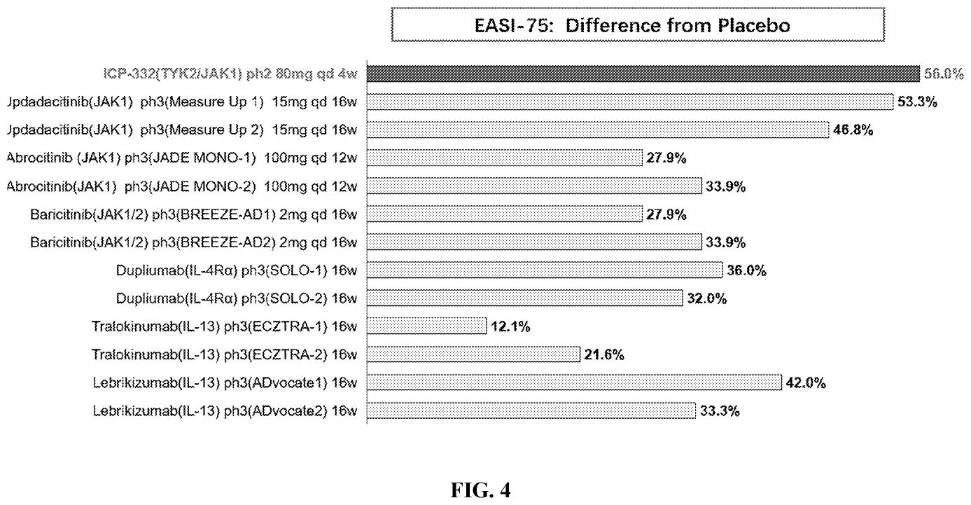

FIG. 4 shows EASI-75 (difference from placebo) of ICP-332 versus those of other drugs for treating AD as monotherapy. The treatment period for ICP-332 was 4 weeks, while it was 12 or 16 weeks for other drugs with their approved recommended doses from phase III studies. The data of other drugs are based on available published literature. Although the comparison is not head-to-head, our results show that, comparing with the public available data of other drugs, ICP-332 had the best EASI-75 value over placebo.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed is:

1. A method for treating atopic dermatitis, comprising the step of orally administering to a subject in need thereof Compound A, B, or C

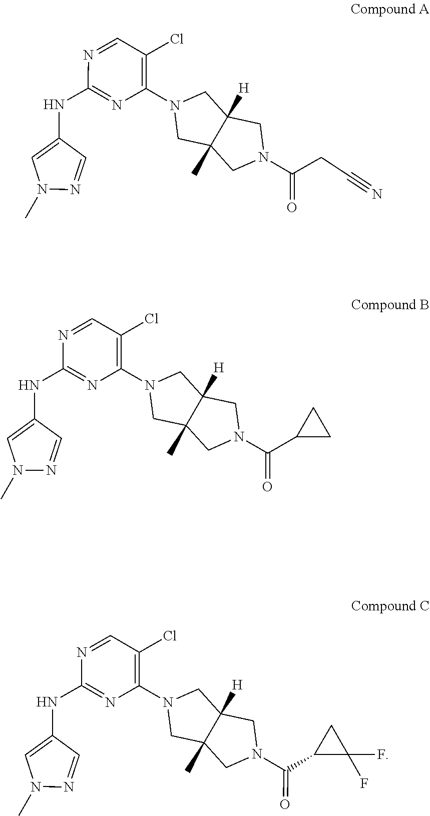 [SMILES: Cn1cc(Nc2ncc(Cl)c(N3C[C@@H]4CN(C(=O)CC#N)C[C@]4(C)C3)n2)cn1 ; Cn1cc(Nc2ncc(Cl)c(N3C[C@@H]4CN(C(=O)C5CC5)C[C@]4(C)C3)n2)cn1 ; Cn1cc(Nc2ncc(Cl)c(N3C[C@@H]4CN(C(=O)[C@@H]5CC5(F)F)C[C@]4(C)C3)n2)cn1]

2. The method of claim 1, wherein the subject is treated with 50-150 mg/day of Compound A, B, or C.

3. The method of claim 1, wherein the subject is treated with Compound A.

4. The method of claim 3, wherein the subject is treated with about 80-120 mg of Compound A once a day.

5. The method of claim 1, wherein the subject is treated with Compound B.

6. The method of claim 5, wherein the subject is treated with about 80-120 mg of Compound B once a day.

7. The method of claim 1, wherein the subject is treated with Compound C.

8. The method of claim 7, wherein the subject is treated with about 80-120 mg of Compound C once a day.
